(19) United States
(12) Patent Application Publication
(10) Pub. No.: US 2016/0150964 A1
(43) Pub. Date: Jun. 2, 2016

(54) COMPREHENSIVE MEASURING METHOD OF BIOLOGICAL MATERIALS AND TREATMENT METHOD USING BROADLY TUNABLE LASER

(71) Applicant: SWAT, INC., Los Angeles, CA (US)

(72) Inventors: Kazuo TSUBOTA, Tokyo (JP); Satoshi WADA, Saitama (JP); Atsushi SHINJO, Kanagawa (JP)

(73) Assignee: SWAT INC., Los Angeles, CA (US)

(21) Appl. No.: 15/015,618

(22) Filed: Feb. 4, 2016

Related U.S. Application Data
(62) Division of application No. 13/661,988, filed on Oct. 26, 2012, now abandoned.
(60) Provisional application No. 61/552,773, filed on Oct. 28, 2011.

(51) Int. Cl. A61B 5/00; A61N 5/06

(52) U.S. Cl. CPC A61B 5/0075; A61N 5/0624; A61N 2005/067

(57) ABSTRACT

A noninvasive method for measuring biological materials which is configured to enable an immediate diagnosis is used in an analytical absorption spectroscopy apparatus including a broadly tunable infrared laser oscillation device, a photodetection device, and an analysis device. The method applies an infrared ray to an analyte organism while tuning the wavelength from the laser oscillation device with or without the ray going through a nonlinear optical device. The photodetection device detects a reflected beam, a transmitted beam, or a scattering beam from the organism or ultrasound generated within the organism by using the detection device. The analysis device analyzes a signal input by the photodetection device.

### CROSS REFERENCE TO RELATED APPLICATIONS

The present application is a division of U.S. application Ser. No. 13/661,988, filed on Oct. 26, 2012, which claims the benefit of U.S. Provisional Application No. 61/552,773, filed on Oct. 28, 2011. The entire contents of each of U.S. application Ser. No. 13/661,988 and U.S. Provisional Application No. 61/552,773 are hereby incorporated by reference in their entirety.

### BACKGROUND OF THE INVENTION

1. Field of the Invention

The present invention relates to a noninvasive method for comprehensively measuring biological materials with a broadly tunable laser in an infrared region. In addition, the present invention relates to a method for treating diseases occurring in organisms by applying optical stimulus in an infrared region to biological materials by using a broadly tunable laser.

2. Description of the Related Art

Electromagnetic waves in the infrared regions are very important in relation to activities of organisms. More specifically, electromagnetic waves in the infrared regions are useful in examining conditions or states of organisms. Visible rays range from 350 nm to 780 nm. Infrared rays have wavelengths distributed in a range of approximately 0.7 μm to 1 mm (1,000 μm). More specifically, such infrared rays are classified into near infrared rays, mid-infrared rays, and far infrared rays according to their wavelengths, although different academic societies or institutes have presented slightly different definitions of respective wavelength sections. Near infrared rays are electromagnetic waves having wavelengths in the range of approximately 0.7 to 2.5 μm. Furthermore, near infrared rays have wavelengths close to those of red visible light. Mid-infrared rays are electromagnetic waves having wavelengths in the approximate range of 2.5 to 10 μm. In particular, mid-infrared regions are referred to as “finger-print regions”, in which absorption spectra inherent to materials are presented. Accordingly, mid-infrared rays are used in identifying chemical materials. In addition, far infrared rays are electromagnetic waves having wavelengths in the approximate range of 10 to 1,000 μm.

In general, when infrared rays are applied to materials, molecules constituting the materials absorb energy of the light. As a result, the state of quantized vibration or rotation changes. The energy necessary for the excitation of the above-described vibration and rotation of molecules differs according to the chemical structure of the molecules. Accordingly, infrared absorption spectra, which are obtained in a graph in which the wave numbers of the irradiated infrared rays are taken on the horizontal axis and the absorbance is taken on the vertical axis, take shapes specific to the molecules. With the infrared absorption spectra having the shape specific to the molecules, the structures of the target materials can be determined. In addition, infrared spectra of the same molecules may slightly differ according to the temperature and their environment (i.e., if the molecules can freely move, if they have adhered to a surface of any organ or tissue, and the like). In other words, surface structures of the materials can be determined according to the change in the infrared spectra of the constituent molecules.

The sensitivity of infrared spectroscopy, which utilizes the above-described characteristics of infrared spectra, is higher than those of other spectrometries. Accordingly, the infrared spectroscopy is commonly used in physical chemistry, which often uses gaseous samples or minute quantities of samples.

Therefore, infrared rays having the above-described characteristics are known to be useful in determining biological materials and the conditions thereof. For example, Japanese Patent No. 4602765 discusses a method that utilizes rays having frequencies in the near infrared region in determine conditions of vascular walls. Japanese Patent Application Laid-Open No. 2007-236734 discusses an apparatus that measures the concentrations of biological components according to measurement results of infrared rays from the ear drum.

The above-described conventional techniques measure specific characteristics of specific regions of organisms.

### SUMMARY OF THE INVENTION

The present invention is directed to a noninvasive method for comprehensively determining and measuring the presence or absence and conditions of various biological materials by utilizing a recently developed broadly tunable laser controlled by a computer.

According to an aspect of the present invention, a noninvasive method for comprehensively measuring biological materials, which is used in an analytical absorption spectroscopy apparatus including a broadly tunable infrared laser oscillation device controlled by a computer, a photodetection device, and an analysis device, includes applying an infrared ray to an analyte organism while tuning a wavelength of a ray emitted from the laser oscillation device, detecting a reflected beam, a transmitted beam, or a scattering beam from the organism or ultrasound generated within the organism by using the photodetection device, and analyzing a signal input by the photodetection device by using the analysis device.

Values, contents, quantities, counts, and the like used by the method of the present invention include the following measurement values representing conditions of organisms (the health statuses) that are targets of measurement, respective contents of one or more kinds of materials, a total content of a group of one or more kinds of materials, or the quantities of cells or germs present or existing in vivo or on a surface of organisms. However, the values, contents, qualities, counts, and the like used by the method of the present invention are not limited to those described below. More specifically, the method of the present invention uses values, contents, qualities, and the like in relation to Escherichia coli (E. coli) or various kinds of pathogenic germs (i.e., bacteria, fungi, and acanthamoeba), viruses, malignant tumors or malignant neoplasms (e.g., scirrhous carcinoma), various kinds of lesions, traumas, total protein (T.P.), albumin, albumin-globulin (A/G) ratio (bromocresol green (BCG)), total bilirubin, direct reacting bilirubin, aspartate aminotransferase (AST) (glutamic oxaloacetic transaminase (GOT)), alanine transaminase (ALT) glutamic pyruvic transaminase (GPT)), lactate dehydrogenase (LD or LDH), cholinesterase (Ch-E), alkaline phosphatase (ALP), gamma-glutamyl transpeptidase (γ-GT or γ-GTP), creatine kinase (CK (creatine phosphokinase (CPK)), vitamin D:25 (OH) D3, serum iron, unsaturated ironbinding capacity (UIBC), ferritin, serum copper, serum zinc, Na, Cl, K, Ca, inorganic phosphorus, magnesium, amylase, urea nitrogen (blood urea nitrogen (BUN) (blood nitrogen of urea origin)), creatinine, uric acids, blood glucose, hemoglobin A1C, insulin, C-reactive protein (CRP), total prostate specific antigen (PSA), dehydroepiandrosterone sulfate (DHEA-S), free testosterone, somatomedin, total cholesterol, high-density lipoprotein cholesterol (HDL-cho), low-density lipoprotein cholesterol (LDL-cho), neutral fats, total homocysteine, fatty acid 4-fractions, arachidonic acid (AA), eicosapentaenoic acid (EPA), docosahexaenoic acid (DHA), white cell count (WCC), red blood cell count (RBC), hemoglobin amount, hematocrit, mean corpuscular volume (MCV), mean corpuscular hemoglobin (MCH), mean corpuscular hemoglobin concentration (MCHC), platelet count, a series of measurement values used in blood tests (neutro, eosino, baso, mono, lympho, and the like), and contents of mercury, lead, zinc, aresevic, and the like. In addition, numerical values that represent the status of immunoreactivity and the like can be measured by the present invention. The pathogens that are the objects of determination by the method of the present invention include Staphylococcus aureus (SA), coagulase-negative staphylococci (CNS), corynebacteria, serratia bacteria, pneumococci, Staphylococcus epidermidis, Moraxella, Pseudomonas aeruginosa, streptococci, fungus aspergillus, candida, and viruses, such as varicella-zoster virus (VZV) or herpes simplex virus (HSV), and the like.

According to an aspect of the present invention, contents of materials having a complex structure, such as glucose present in organisms, can be measured after noises are eliminated because the present invention uses broad-band infrared rays. In other words, according to an aspect of the present invention, it is enabled to perform measurement without requiring the separation of samples from organisms. In addition, according to an aspect of the present invention, although the presence of water may produce a noise in measuring numerical values that represent conditions of organisms, it is enabled to effectively eliminate adverse effects from the noise because appropriate broad-band infrared rays are used. In addition, according to an aspect of the present invention, it is enabled to identify materials and the like ranging over a plurality of finger-print regions from among the measuring objects described above and to measure the content of respective materials.

Furthermore, according to an aspect of the present invention, the organisms that are the objects of the measurement by the method of the present invention include animals and plants in addition to humans. Regions of humans and animals to be measured by the method of the present invention generally include body surfaces and intracorporeal mucosa. The body surfaces include the skin, the external ear, and the cornea in addition to the retina and the retinal blood vessels. Note that the retina and the retinal blood vessels do not actually exist as body surface regions but are herein included in the body surfaces because they can be observed noninvasively from outside the body. In other words, the objects measured by the method of the present invention include mucosae inside the oral cavity or nasal cavity. In addition, the measuring objects of the method of the present invention include mucosae of the esophagus, stomach, duodenum, intestinum, and the like of the body, which can be measured by using an optical fiber that guides light and/or a small-size light source and photodetection device.

For the computer-controlled broadly tunable laser oscillation device, an electronic tunable laser oscillation device which is capable of controlling the wavelength of light in the infrared region at a high speed by using a computer program can be used. In the present invention, the term “high speed” refers to the performance of continuously tuning the wavelength in the entire tunable region at a cycle of 1 mS or less, such as 1 μS. The conversion of wavelength can be implemented by continuous tuning. Alternatively, pulses of different wavelengths ranging from 500 to 5,000 nm (in particular, 700 nm, 1,000 nm, or 2,000 nm) can be output. At the present time, an acoustooptic device, which is known as an acousto optic tunable filter (AOTF), is inserted into a laser resonator. More specifically, a transmission grating can be generated in an optical crystal provided in an AOTF device by generating compression waves by applying acoustic waves to the optical crystal. In the transmission grating, intervals between acoustic waves are changed according to an ultrasonic frequency controlled by a computer. With the changeable interval between the acoustic waves, the wavelength of diffraction ray can be selected. Because the wavelength of the diffraction ray can be selected as described above, a laser having a specific wavelength can be generated within an optical resonator. More specifically, with the tunable infrared laser, electronic wavelength tuning in the wavelength range of 680 to 1,100 nm is achieved.

Such a high-speed and broadly tunable laser cannot be achieved by a method that converts a laser beam by the conventional nonlinear wavelength conversion. In the nonlinear wavelength conversion, in order to tune a wavelength, it is necessary to change the angle of a nonlinear optical crystal together with the wavelength because it is required to satisfy the photon momentum conservation law, which is known as a phase matching condition. To change the angle of a nonlinear optical crystal together with the wavelength, mechanical devices are utilized to restrict the speed and the accuracy of tuning the wavelength. In addition, the range of tunable wavelength is limited.

Furthermore, by generating two acoustic waves on the AOTF, two laser rays whose wavelength can be tuned independently from each other can be obtained from one laser light source. When the laser light emitted from the laser light source is incident to a special nonlinear optical crystal, a laser beam corresponding to the difference between two frequencies can be freely generated. The wavelength region of the generated laser beam ranges from several microns to the terahertz region.

### BRIEF DESCRIPTION OF THE DRAWINGS

FIG. 1 is a conceptual diagram illustrating an example of an analytical absorption spectroscopy apparatus used in a first exemplary embodiment of the present invention.

FIG. 2 is a conceptual diagram illustrating an example of an analytical absorption spectroscopy apparatus used in a second exemplary embodiment of the present invention.

### DESCRIPTION OF THE EMBODIMENTS

Exemplary embodiments of the present invention will be described in detail below.

According to a first exemplary embodiment, the present invention is a noninvasive method for comprehensively measuring biological materials. The noninvasive method for comprehensively measuring biological materials according to the first exemplary embodiment is used in an analytical absorption spectroscopy apparatus including a broadly tunable infrared laser oscillation device controlled by a computer, a photodetection device, and an analysis device. More specifically, the noninvasive method for comprehensively measuring biological materials according to the first exemplary embodiment includes a step for applying an infrared ray to an analyte organism while tuning the wavelength of the ray emitted from the laser oscillation device, a step for detecting a reflected beam, a transmitted beam, or a scattering beam from the organism or ultrasound generated within the organism by using the photodetection device, and a step for analyzing a signal input by the photodetection device by using the analysis device.

The basic principle of the apparatus according to an embodiment of the present invention is to conduct a qualitative or quantitative evaluation of a target region of an organism according to a result of measurement of a characteristic of a reflected beam, a transmitted beam, or a scattering beam from the organism, or ultrasound generated in the organism by applying light having a specific wavelength in the infrared region to the target region of the organism. More specifically, proteins and sugar chains of fungi, viruses, and malignant tumors have optical characteristics (i.e., spectral characteristics) inherent to respective materials. Such spectra are caused by vibrational-rotational transition of molecules. Accordingly, the vibrational-rotational transition of molecule can be caused particularly by precisely measuring absorption spectra or Raman spectra of the molecules in the infrared ray region.

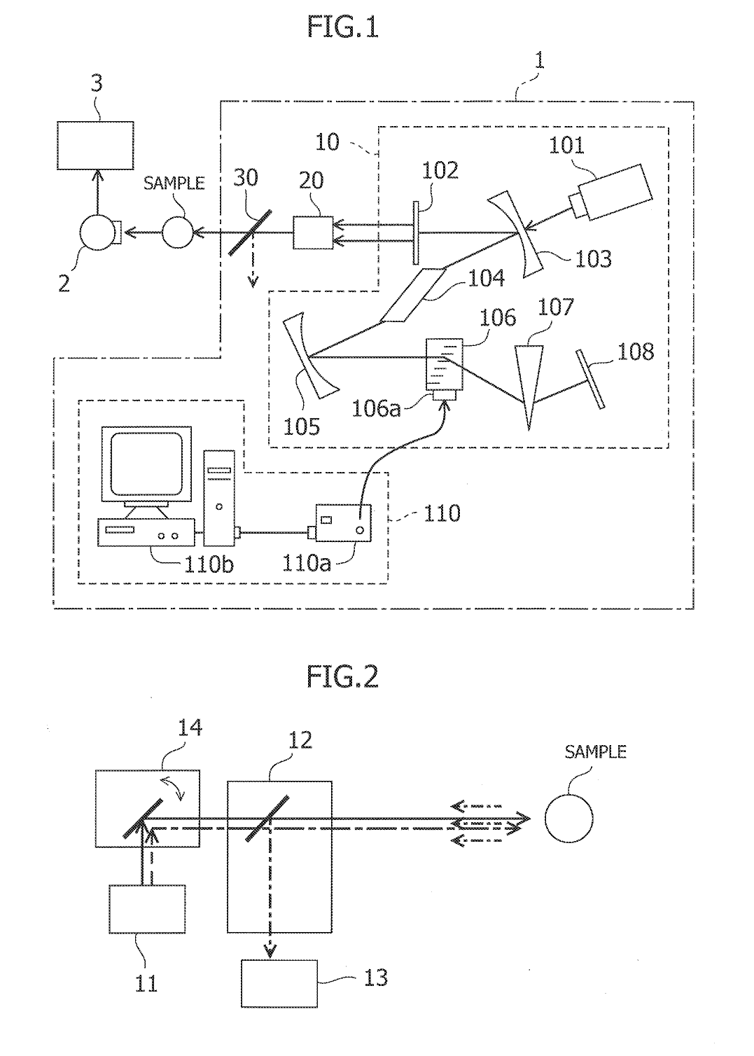

FIG. 1 is a conceptual diagram illustrating an example of an analytical absorption spectroscopy apparatus which can be used in the first exemplary embodiment. The analytical absorption spectroscopy apparatus includes a tunable infrared laser oscillation device 1, a photodetection device 2, and an analysis device 3.

The tunable infrared laser oscillation device 1 is configured to irradiate the cornea of an analyte with a light beam whose wavelength can be broadly tuned. The present invention is greatly useful in a point that the present invention uses the tunable infrared laser oscillation device 1 having the function described above, which has not been conventionally commercialized or used in medical scenes.

In the example illustrated in FIG. 1, the tunable infrared laser oscillation device 1 includes a neodymium-doped yttrium lithium fluoride (Nd:YLF) pumped laser 101, an output mirror 102, a high-reflectance mirror 108, two concave mirrors 103 and 105, a Ti:Al₂O₃ laser crystal 104, a photoacoustic optical crystal 106, a diffraction ray correction prism 107, a nonlinear optical crystal AgGaS₂ 20, and a Ge filter 30. Among the components of the tunable infrared laser oscillation device 1 described above, the output mirror 102, the high-reflectance mirror 108, the two concave mirrors 103 and 105, the Ti:Al₂O₃ laser crystal 104, the photoacoustic optical crystal 106, and the diffraction ray correction prism 107 are included in a laser resonator 10.

More specifically, in the laser oscillation device 1, the laser resonator 10 includes the output mirror 102 having a predetermined permeability and the high-reflectance mirror 108. In the laser device 10, the concave mirror 103, which is one of the two concave mirrors, the Ti:Al₂O₃ laser crystal 104, which is a tunable laser, the concave mirror 105, which is the other concave mirror, the photoacoustic optical crystal 106, which is a crystal for selecting the wavelength, and the diffraction ray correction prism 107 are serially provided in this order from the output mirror 102 towards the high-reflectance mirror 108 in a z-fold arrangement. The Ti:Al₂O₃ laser crystal 104 is located between concave surfaces of the two concave mirrors 103 and 105. More specifically, the two concave mirrors 103 and 105 are respectively located at the vertex of two corners of the z-like shape of the arrangement.

Furthermore, a piezoelectric element 106a is impregnated to the photoacoustic optical crystal 106 as an acoustic wave input device. The piezoelectric element 106a is connected to a radio frequency (RF) modulation voltage power source (hereinafter simply referred to as the “RF power source”) 110. The piezoelectric element 106a is driven by the RF power source 110. The RF power source 110 can include an RF amplifier 110a, which is connected to the piezoelectric element 106a, and a computer 110b, which is connected to the RF amplifier 110a. The computer 110b includes a synthesizer board, which is inserted inside thereof, and software installed thereto. The synthesizer board is configured to generate an RF of a desired frequency and intensity determined by the computer 110b. The software is configured to enable high-speed wavelength sweeping and random wavelength selection. For example, the computer 110b can perform the high-speed wavelength tuning at 2 mS. The light intensity can be serially selected within a range from 0 to 100%. In addition, two different frequencies can be simultaneously applied as the RF.

When the piezoelectric element 106a is driven by the RF power source 110 and the piezoelectric element 106a to generate distortion on the piezoelectric element 106a, an acoustic wave having a frequency corresponding to the distortion is input to the photoacoustic optical crystal 106 according to the distortion of the piezoelectric element 106a. The diffraction ray correction prism 107 is configured to emit the diffraction ray exit from the photoacoustic optical crystal 106 invariably in a fixed direction regardless of the wavelength. The high-reflectance mirror 108 is configured to reflect the light emitted from the diffraction ray correction prism 107.

The tunable infrared laser oscillation device 1 having the above-described configuration is configured to excite the Ti:Al₂O₃ laser crystal 104 by using a second harmonic of a neodymium:yttrium-aluminum-garnet (Nd:YAG) laser as a laser beam from the Nd:YLF pumped laser 101. In addition, the tunable infrared laser oscillation device 1 is configured to control the frequency of the RF power source 110 based on the wavelength of an outgoing laser beam to be emitted from the output mirror 102 according to the basic principle described above to drive the piezoelectric element 106a. With the above-described configuration, the outgoing beam having the wavelength corresponding to the frequency of the RF power source 110, among the outgoing beams in the wide frequency bands that have been incident to the photoacoustic optical crystal 106 and emitted from the Ti:Al₂O₃ laser crystal 104, is diffracted in a predetermined direction and emitted from the photoacoustic optical crystal 106 as a diffraction ray. Furthermore, the diffraction ray diffracted in the predetermined direction and emitted from the photoacoustic optical crystal 106 is incident to the diffraction ray correction prism 107 and is emitted in a fixed direction. The two concave mirrors 103 and 105 function as control elements for controlling a mode size of the laser in a laser medium. The ray emitted from the diffraction ray correction prism 107 is reflected by the high-reflectance mirror 108. The reflected ray oscillates inside the laser resonator 10.

In the above-described manner, the ray having a wavelength corresponding to the frequency of the RF power source 110 only is amplified to generate oscillation of the laser and the outgoing laser beam having the wavelength can be emitted from the laser resonator 10. If the Ti:Al₂O₃ laser crystal 104 is used as illustrated in FIG. 1, the outgoing laser beam emitted from the RF power source 110 can be controlled to have a wavelength ranging from 700 to 1,100 nm, an output ranging from 0 to 300 mW, and a repetition frequency of 1 kHz. The pulse width of the outgoing laser beam from the laser resonator 10 can be controlled from a sub-ps pulse width to a continuous pulse width. Note that if Cr:ZeSe is used as the tunable laser crystal, the oscillation wavelength can be set within the range of 1,900 to 3,100 nm.

In addition, when two different frequencies are simultaneously applied as the RF, coaxial oscillation of the lasers in two different wavelengths can be obtained. In the apparatus according to the present exemplary embodiment, it is useful to generate oscillation of the laser in the tunable region of the laser medium.

At a subsequent stage of the output mirror 102 of the laser resonator 10, the nonlinear optical crystal 20, such as BBO crystal ((3-barium borate), and the Ge filter 30 are provided in this order.

The nonlinear optical crystal 20 is a device capable of extending the wavelength region of the outgoing laser beam to a desired wavelength region. In the present exemplary embodiment, a nonlinear optical crystal can be used for rays having two different wavelengths obtained coaxially from the laser resonator 10 to generate a difference frequency. For the nonlinear optical crystal, β-BaB₂O₄ (BBO crystal), LiB₃O₅, KTiOPO₄, LiNbO₃, MgO:LiNbO₃, AgGaS₂, and the like can be used. Among these, BBO and AgGaS₂ are particularly useful. If AgGaS₂ is used as the nonlinear optical crystal and if the phase matching angle is fixed, the wavelength can be tuned by 2 to 3 μm. In addition, by selecting a plurality of phase matching angles, continuous or discrete mid-infrared coherent light sources each having a tunable wavelength region of 2 to 3 μm can be constituted. More specifically, if AgGaS₂ having the phase matching angle of 45 degrees, AgGaS₂ having the phase matching angle of 50 degrees, or AgGaS₂ having the phase matching angle of 55 degrees is used, a continuously tunable laser beam in the tunable wavelength region of 5 to 7.5 μm, 7 to 9 μm, or 9 to 12 μm, respectively, can be obtained. As a result, a continuously tunable laser beam having a tunable wavelength region of 5 to 12 μm in total can be obtained.

The Ge filter 30 is configured to separate a mid-infrared ray obtained by the difference frequency generated by the nonlinear optical crystal 20 from the wavelength from the Ti:Al₂O₃ laser crystal 104. As a result, it is enabled to selectively irradiate the organism, which is the target of the measurement, with the mid-infrared ray only as the outgoing laser beam from the tunable infrared laser oscillation device 1.

The photodetection device 2 is configured to detect a reflected beam, a transmitted beam, or a scattering beam from the measuring-object organism, which is a beam that has been emitted from the tunable infrared laser oscillation device 1, or ultrasound generated within the organism. Furthermore, the photodetection device 2 is configured to transmit information about the intensity of the detected light beam or ultrasound to the analysis device 3 in real time. A wavelength detection region that is the subject of detection by the photodetection device 2 corresponds to the tunable region of the laser oscillation device 1. For example, it is useful if the photodetection device 2 has the wavelength detection region in a range of approximately 5 to 10 μm. For a detector included in a detection unit, an infrared detector, such as an HgCd detector (spot detector) or a non-cooling type bolometer (two-dimensional bolometer), can be used. For a detection area, if the measurement is conducted by spot detection, it is useful if the detection area of a diameter of about 10 μm, for example, is set. On the other hand, if the measurement is conducted by area detection, it is useful if the detection area of a diameter of about 1 cm, for example, is set.

The analysis device 3 is configured to analyze the spectrum acquired by the photodetection device 2 and provide information about the region of the organism that is the measuring object. A computer connected to the photodetection device 2, for example, can be used as the analysis device 3. It is useful if the analysis device 3 has a database of wavelength characteristics of pathogens existing in various kinds of biological materials and organisms. With this configuration, the present exemplary embodiment can remove noises from the obtained spectrum and acquire desired information about the measured region of the organism.

If a plurality of materials exists in region of the organism that is the measuring object, various types of multivariate analysis methods and chemometric methods can be used as the method that analyzes the materials separated from one another. Note that the term “chemometric(s)” refers to a method for providing information important in interpreting experimental results by processing and converting data acquired by an experiment into a format with which the data can be more easily understood in terms of its structure by performing dimensional compression, visualization, regression, discrimination, classification, and the like on the experimentally acquired data by carrying out mathematical science operations, statistic operations, machine learning, pattern recognition, data mining, and the like.

The analysis device 3 can further include a display device and an output device configured to display and output a result of the analysis.

A measurement method according to the first exemplary embodiment of the present invention is a real-time noninvasive measurement method using the above-described diagnostic apparatus (analytical absorption spectroscopy apparatus). More specifically, the measurement method according to the first exemplary embodiment of the present invention includes a step for applying a tunable infrared laser to an analyte, a step for detecting a wavelength characteristic of a reflected beam, a transmitted beam, or a scattering beam from the test object, and a step for acquiring a result of the measurement according to the detected wavelength characteristic.

The measurement method according to the present exemplary embodiment is capable of noninvasively measuring various types of numerical values that represent the presence or absence and states and conditions of germs and materials in an analyte and the health status of the analyte in real time by directly applying a tunable infrared laser to the analyte without requiring any sampling steps. Mammals can be used as the analyte. Humans, pet animals, or livestock are usually used as the analyte. If humans are used as the analyte, the present exemplary embodiment can be applied regardless of the age or pathology of the human analyte. More specifically, because the measurement can be finished within a short period of time according to the present exemplary embodiment, the present exemplary embodiment is useful if the analyte is a child who cannot keep still or an animal.

The step for detecting a reflected beam, a transmitted beam, or a scattering beam from the organism or ultrasound generated within the organism can be implemented by the photodetection device 2.

The step for measuring the health status of an analyte according to the detected wavelength characteristic of the reflected beam, transmitted beam, or scattering beam or the ultrasound generated within the organism can be implemented by the analysis device 3, which is included in the diagnosis apparatus described above. The pathogen can be identified by comparing acquired wavelength characteristic data with wavelength characteristic data accumulated in a database of the analysis device 3 that stores wavelength characteristics of various kinds of materials, germs, or cells, for example.

The determination of a pathogen can be implemented by carrying out spectrum analysis by an already known method. Furthermore, if a plurality of kinds of pathogens exists in the analyte, the present exemplary embodiment can identify and determine a specific pathogen or cell and measure various numerical values that represent the condition of the organism and the content of a chemical matter.

According to the method by the first exemplary embodiment, the health status of the analyte can be noninvasively measured in real time. More specifically, the present exemplary embodiment can determine the inner condition of a capillary of a finger of an analyte, for example. To paraphrase this, the present exemplary embodiment can easily determine values or levels of various kinds of biological materials, such as the blood glucose level, the cholesterol level, the fat level, or the enzyme level immediately after the analyte has taken food and drink. In addition, according to the measurement method of the present exemplary embodiment, cholesterol that has adhered to a blood vessel can be discriminated from cholesterol that flows inside the blood vessel without adhering thereto and the measurement can be performed for cholesterol in respective states. Furthermore, according to the comprehensive measurement method of the first exemplary embodiment, the reaction of biological materials can be chronologically observed. More specifically, the behavior of an antigen adhering to a specific cell can be observed. To paraphrase this, an immunization activity can be observed in real time.

A second exemplary embodiment of the present invention relates to a method for determining the health status of an analyte, which uses an apparatus equipped with a confocal measurement system using the coherent anti-Stokes Raman spectroscopy (hereinafter simply referred to as the “CARS”).

In the coherent anti-Stokes Raman spectroscopy, when a pumped laser (Ep field strength) and a laser having a wavelength corresponding to the wavelength of the Stokes line (Es field strength) are simultaneously applied to a material, polarization of a product of an expression EsEpEp, which is third-order nonlinear polarization, is generated. The polarization vibrates at a short wavelength, which is short by an amount equivalent to the Raman shift amount inherent to the material. More specifically, the polarization compulsorily generates an anti-Stokes line by the strong electric field generated by the laser. Because a signal to be obtained is proportional to the density of the material, the method can identify the density of the material. The above-described technique has recently received attention as a useful spectrometry for materials because it can acquire information about the measured material only according to the difference between two lasers.

In the present exemplary embodiment, the method identifies a target pathogen according to wavelengths of two lasers utilized on a probe and a signal strength acquired from the pathogen by utilizing a laser scanning confocal microscope system, which is utilized in the field of biology. In the confocal measurement system using the CARS, a pumped laser for Raman scattering and a laser for the Stokes line are prepared. By simultaneous application of the two lasers, nonlinear polarization necessary for the CARS spectrometry is excited. The necessary laser output is likely to be lower than that in the case of usual Raman scattering. In addition, the signal strength to be acquired is likely to be higher than that in usual Raman scattering by orders of magnitude. The output to be acquired is theoretically proportional to the density. A quantitative measurement can be performed according to a determined correlation between the concentration and the scattering strength.

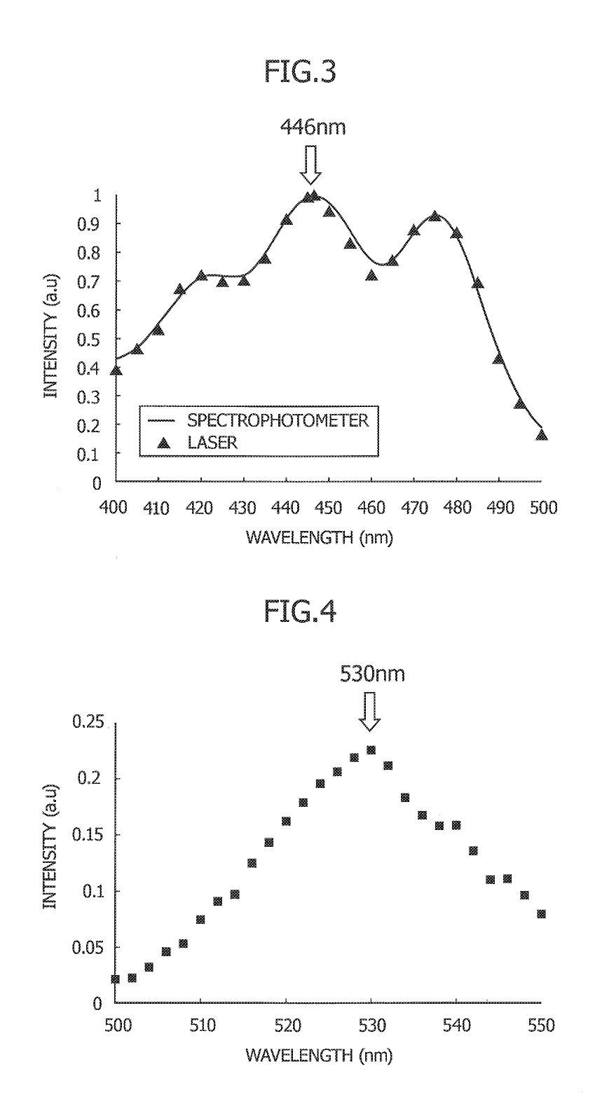

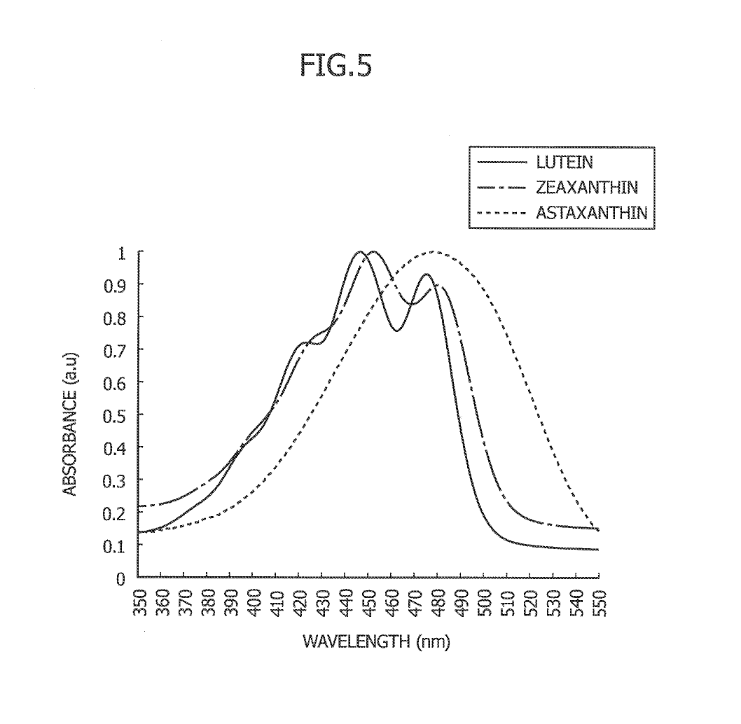

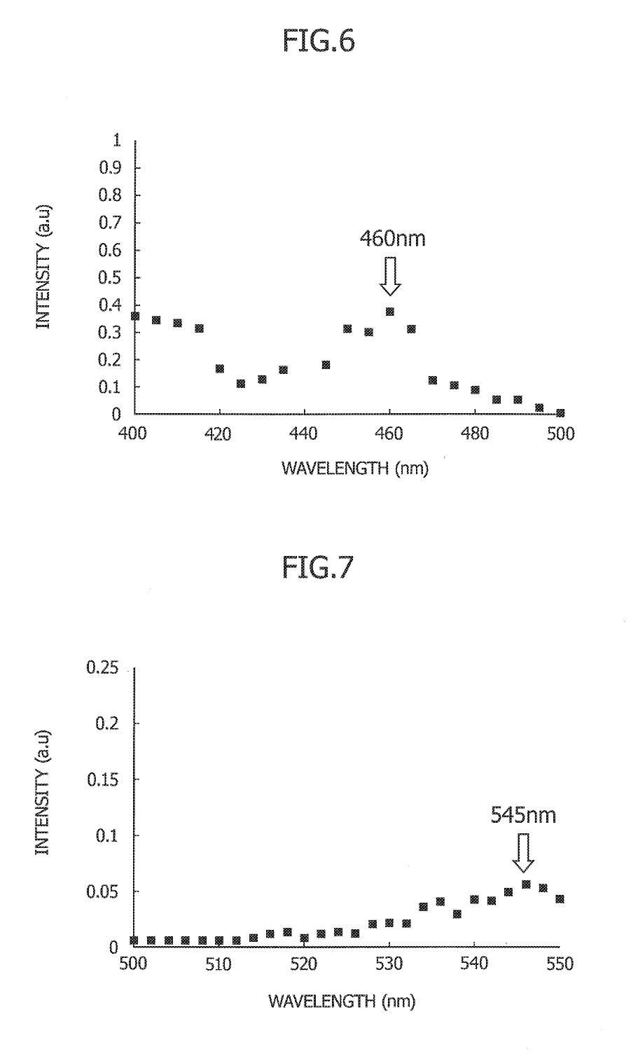

FIG. 2 is a conceptual diagram illustrating an example of an apparatus which can be used in the second exemplary embodiment. The confocal measurement apparatus using the CARS according to the second exemplary embodiment includes a dual wavelength laser oscillator 11, a confocal optical unit 14, a photodetection device 12, and an analysis device 13.

The dual wavelength laser oscillator 11 can use a pulse output fiber laser light source. In particular, it is useful to use a visible dual wavelength pulse laser (ps-ns) having an in-plane resolution of 100×100 μm and a depth resolution of 20 μm as the dual wavelength laser oscillator 11. Alternatively, a laser oscillator capable of simultaneously emitting lasers of two different wavelengths can be constituted by using the laser resonator 10 used in the first exemplary embodiment described above. In this case, the laser oscillator can have a simple configuration including an optical element and a filter instead of the nonlinear optical crystal 20 and the Ge filter 30 used in the first exemplary embodiment.

The confocal optical unit 14 is an optical system configured to securely obtaining the focus in the direction of depth of the cornea. For the confocal optical unit 14, an optical system usually used in a confocal microscope can be used. More specifically, components such as a pinhole and an optical filter for selecting a signal beam are included in the confocal optical unit 14 at the focal point and the confocal point of an object lens in the measuring object. For a particularly useful specification of the filter included in the confocal optical unit 14, it is useful to use a filter having a characteristic of reflecting an incident ray in the wavelength region of 700 to 1,000 nm and transmitting the incident ray in the wavelength region of 550 to 650 nm, according to an exemplary result of measurement of lutein.

For the photodetection device 12, a detection device commonly used in CARS spectrometry systems can be used. More specifically, the photodetection device 12 includes components such as a filter having a characteristic of reflecting an incident ray having the wavelength of an excitation ray or Stokes ray and transmitting an incident ray having the wavelength of an anti-Stokes ray of a signal beam. For a particularly useful specification of the filter included in the photodetection device 12, according to an exemplary result of measurement of lutein, it is useful to use a filter having a characteristic of reflecting an incident ray in the wavelength region of 700 to 1,000 nm of an excitation ray or Stokes ray and transmitting the incident ray in the wavelength region of 550 to 650 nm of an anti-Stokes ray of a signal beam.

The confocal measurement system that uses the CARS described above can be manufactured and implemented by using any commonly used conventional technique. It is useful if the confocal measurement apparatus further includes an irradiation control device, which is configured to control the application of beam according to characteristics of a region to be measured of the analyte.

The analysis device 13 is utilized to analyze CARS spectra acquired by the confocal measurement system that uses the CARS and is configured to provide information about the analyte. A beam scan system can be connected to the photodetection device 12. The analysis device 13 is configured to convert the acquired CARS spectra into three-dimensional images to spatially resolve the measuring object. In addition, the analysis device 13 is configured to remove a noise of any kind.

Now, the second exemplary embodiment of the present invention will be described in detail below in terms of its measurement method. The measurement method according to the second exemplary embodiment includes a step for sweeping a wavelength in a fixed range by applying a dual wavelength pulse laser to an analyte, a step for obtaining CARS spectra from the analyte, and a step for calculating a numerical value according to the obtained spectra.

In the measurement method according to the present exemplary embodiment, an analyte similar to that used in the first exemplary embodiment described above can be used.

The confocal measurement system that uses the CARS can implement the step for sweeping the wavelength in a fixed range by irradiating the cornea of an analyte with a dual wavelength pulse laser and the step for obtaining CARS spectra from the cornea. As a sweeping condition, it is required that the analyte is irradiated with lasers having wavelengths of a Stokes ray and excitation ray according to the Raman shift amounts of bacteria and viruses, which are measurement subjects that have been previously measured. In addition, as a spectroscopic condition, it is required to provide an optical system capable of selectively detecting a ray having a wavelength corresponding to an anti-Stokes ray.

By using an apparatus equipped with the confocal measurement system that uses the CARS, information about the analyte in the direction of the depth can be particularly acquired. Accordingly, the apparatus can be useful in carrying out the measurement of an analyte to determine the condition of inner distribution.

The first and the second exemplary embodiments of the present invention have the following collateral effect. More specifically, in a small-size hospital and the like in which diagnostic techniques or apparatuses are not introduced to their medical scenes, inevitable diagnostic treatments are performed based on findings on the patients' conditions. In other words, in the hospitals and the like described above, doctors may conduct treatments by approximately determining the observed effects of administered drugs. In this case, drugs for different targets may be administered and there is a threat of patients' conditions getting worse. Under such circumstances, the present invention enables a prompt measurement of biological materials. With the diagnostic method that can be established by the present invention, the otherwise possible increase in the medical expenses borne by patients due to worsened disease condition can be prevented.

A third exemplary embodiment of the present invention is a method for performing a treatment of an analyte by using a broadly tunable laser according to the result of measurement obtained by the first or the second exemplary embodiment. The treatment method includes a step for selecting the wavelength and the intensity of the laser beam according to the measurement result and a step for applying the selected laser beam to the analyte organism.

By applying an infrared ray or ultraviolet light having a specific wavelength to pathogenic bacteria or materials, the pathogenic bacteria or materials can be broken or detoxicated.

It is known that an infrared ray having a wavelength in a specific range is effective in both killing pathologic bacteria and inhibiting activities of enzymes. The method according to the present exemplary embodiment can apply the infrared ray having the above-described effect. The method according to the present exemplary embodiment can break cancer cells without affecting normal cells that exist peripheral to the cancer cells. In addition, near infrared rays having an wavelength in a range from 700 to 1,000 nm are known to contribute to activation of mitochondria. By activating various kinds of organs and mitochondria existing in cells, the activity of a specific cell can be increased. Among mitochondria, cytochrome oxidase can be particularly used as a target of the activation. The intensity of infrared beams necessary in this case can be appropriately determined by a person skilled in the art. More specifically, the infrared ray can be applied by pinpoint application with a decreased beam width. Furthermore, an infrared ray having a wavelength at which target germs or protein are affected but peripheral tissues are not affected can be applied.

In addition, application of ultraviolet light is known to be effective in breaking cells. The cytoclasis with ultraviolet light can be performed regardless of its wavelength.

The wavelength and the intensity of the laser beam to be applied are selected according to the respective purposes described above and the laser beam of the selected wavelength and intensity is applied to a desired region of the analyte organism by using the tunable infrared laser oscillation device 1 according to the first exemplary embodiment or other infrared laser apparatuses or ultraviolet laser apparatuses used in medical scenes.

According to the present exemplary embodiment, it is enabled to immediately perform the therapy according to a measurement result of the methods of the first and the second exemplary embodiments. Accordingly, the present exemplary embodiment is greatly useful in performing a treatment of diseases that progress rapidly.

### Experimental Results

Experiments were carried out for three carotenoids: lutein, zeaxanthin and astaxanthin. Lutein and zeaxanthin are carotenoids that are present in the retina and macula of eyes. Of about 600 carotenoids found in nature, only lutein and zeaxanthin are present in the macula of the retina. The concentrations of lutein and zeaxanthin in the macula are known to relate to macular degradation or maculopathy. Zeaxanthin is apparently more effective in protecting macula, but lutein is converted in the human body to mesozeaxanthin, which functions similarly to zeaxanthin in macula, and probably useful in protecting the macula. Zeaxanthin is also known to be useful in inhibiting cancer cells in mice. Astaxanthin is also an antioxidant and, like other carotenoids, has self-limited absorption orally and such low toxicity by mouth that no toxic syndrome is known.

FIGS. 3 and 4, respectively, shows the absorption and fluorescence of a lutein solution with a concentration of 1 μmol/l in ethanol. Specifically, FIG. 3 shows an absorption spectrum with a peak at 446 nm. The intensity is in arbitrary units (a.u.). FIG. 4 shows a fluorescence spectrum with a peak at 530 nm. The excitation was done at 446 nm. In order to obtain spectra between 350 nm and 550 nm for these substances, the output from laser oscillation device 1 (in FIG. 1), a Ti:Al₂O₃ tunable laser, with a wavelength range of 700 nm to 1000 nm was frequency doubled using a BBO crystal to obtain the spectral range of 350 nm to 500 nm.

FIG. 5 shows the absorption of lutein, zeaxanthin, and astaxanthin solutions with a concentration of 1 μmol/l in ethanol. In order to compare the peaks of each substance, the intensity is scaled to 1.0.

FIGS. 6 and 7 shows the absorption and fluorescence of a macula sample prepared from a human eye. FIG. 6 is the absorption spectrum with a peak at 460 nm, and FIG. 7 is the fluorescent spectrum with a peak at 545 nm. For these measurements, a human eye was dissected into different parts, and the retina was obtained. The macula portion (about 8 mm×8 mm) was cut out from the retina and washed by 50% glycerol/PBS. The sample was positioned on a slide glass. The absorption and fluorescence excited at 460 nm were measured. In order to measure fluorescence, a spectroscope and a photodetector were positioned at a right angle to the optical path of the coincident ray and a photodetector for absorption measurements.

1-2. (canceled)

3. A noninvasive method for comprehensively measuring biological materials used in a confocal measurement apparatus including a dual wavelength laser oscillation device, a confocal optical unit, a photodetection unit, and an analysis unit and using coherent anti-Stokes Raman scattering spectroscopy, the method comprising:
applying laser beams having two different wavelengths to an analyte organism while tuning the wavelength from the dual wavelength laser oscillation device;
detecting a scattering ray from the organism by using the photodetection unit; and
analyzing a signal from the photodetection unit by using the analysis unit.

4. (canceled)

5. A method for performing a treatment on an analyte organism by using a broadly tunable laser according to a result of the measurement by the comprehensive measurement method according to claim 3, the method comprising:
selecting a laser beam according to the measurement result; and
applying the selected laser beam to the analyte organism.
